(19) United States
(12) Patent Application Publication
(10) Pub. No.: US 2026/0265811 A1
(43) Pub. Date: Sep. 10, 2026

(54) A SURFACE-BASED CRISPR NUCLEIC ACID DETECTION

(71) Applicant: Siemens Healthineers Nederland B.V., Den Haag, Zuid-Holland (NL)

(72) Inventors: Nivedita MITRA, Bangalore (IN); Ramya V M, Bangalore (IN); Emiel PEETERS, Eindhoven (NL); Alexander VAN REENEN, Vught (NL)

(21) Appl. No.: 19/166,317

(22) PCT Filed: Mar. 18, 2024
(86) PCT No.: PCT/EP2024/057100

§ 371 Date: Sep. 17, 2025

(30) Foreign Application Priority Data
Mar. 24, 2023 (WO) PCT/EP2023/057655

(51) Int. Cl. C12Q 1/6825; C12N 9/22; C12N 15/11; C12Q 1/34; C12Q 1/6806; C12Q 1/6823; C12Q 1/6844

(52) U.S. Cl. CPC C12Q 1/6825; C12N 9/226; C12N 15/111; C12Q 1/34; C12Q 1/6806; C12Q 1/6823; C12Q 1/6844; C12N 2310/20; G01N 2333/922

(57) ABSTRACT

The present invention relates to a method for the detection of a target nucleic acid, comprising providing a multitude of nucleic acid detector probes, each being linked to a magnetic bead and tethered to a substrate; detecting the number of magnetic beads linked to the substrate by frustrated total internal reflection (f-TIR); providing a complex of a guidance nucleic acid and a not activated nuclease; activating the nuclease complex by the addition of a target nucleic acid to induce degradation of the nucleic acid detector probe; removing unbound magnetic beads; detecting the number of magnetic beads linked to the substrate by f-TIR; and determining the amount of target nucleic acid in accordance with the ratio of magnetic beads detected. Also envisaged is the use of nucleic acid detector probes and a nuclease complex for the detection of a target nucleic by frustrated total internal reflection (f-TIR) measurement.

### TECHNICAL FIELD

The present invention relates to a method for the detection of a target nucleic acid, comprising providing a multitude of nucleic acid detector probes, each being linked to a magnetic bead and tethered to a substrate; detecting the number of magnetic beads linked to the substrate by frustrated total internal reflection (f-TIR); providing a complex of a guidance nucleic acid and a not activated nuclease; activating the nuclease complex by the addition of a target nucleic acid to induce degradation of the nucleic acid detector probe; removing unbound magnetic beads; detecting the number of magnetic beads linked to the substrate by f-TIR; and determining the amount of target nucleic acid in accordance with the ratio of magnetic beads detected. Also envisaged is the use of nucleic acid detector probes and a nuclease complex for the detection of a target nucleic by frustrated total internal reflection (f-TIR) measurement.

### BACKGROUND

Point-of-care testing (POCT) or bedside testing, is generally understood as medical diagnostic testing at or near the point of care-that is, at the time and place of patient care. Point-of-care diagnostics started as a supplement to standard lab-based diagnostics and has become a very important analysis tool of its own. In particular, the COVID-19 pandemic has shown that there is a growing need for quick, easy-to-operate, reliable, and affordable diagnostic tests and devices at the Point-of-Care (POC).

Throughout Europe and internationally, POCT is used across a variety of settings, including intensive care settings, neonatal and birthing units, operating theatres, general practice, nursing homes, pharmacies, outpatient and off-site clinics and in-home patient care. POCT is typically implemented via Lab-on-chip technologies, which enable different bioassays including microbiological culturing, PCR or ELISA to be used at the point of care. This is often achieved through the use of handheld devices and corresponding test kits. Currently used POC diagnostics are, for example, based on reverse transcriptase loop-mediated isothermal amplification (RT-LAMP) and a CMOS semiconductor platform, which a smartphone for data is coupled to visualization and geolocaliziation (Rodriguez-Manzano et al., 2021, ACS Cent Sci, 7 (2), 307-317). Other approaches include microfluidic technologies which allow analytical steps such as nucleic acid detection in microfabricated channels or in microfluidic chips (Zhang et al., 2016, Analytical Methods, 8, 7847-7867).

However, current point of care nucleic acid tests typically have less than desirable sensitivity and specificity. Furthermore, there are no promising platforms which nucleic acid tests can be performed, which renders an integration and automatization of molecular assays difficult.

There is hence a need for reliable, specific, sensitive, and easily automatable point of care tests for nucleic acids.

### SUMMARY

The present invention addresses this need and provides in a first aspect a method for the detection of a target nucleic acid, comprising the steps: (a) providing a multitude of nucleic acid detector probes, each being linked to a magnetic bead and tethered to a substrate; (b) detecting the number of magnetic beads linked to the substrate by frustrated total internal reflection (f-TIR); (c) providing a complex of a guidance nucleic acid and a not activated nuclease (nuclease complex); (d) activating the nuclease complex of step (c) by the addition of a target nucleic acid, wherein said activation results in the provision of a nuclease activity, to induce degradation of the nucleic acid detector probe; (e) removing unbound magnetic beads; (f) detecting the number of magnetic beads linked to the substrate by f-TIR; and (g) determining the amount of target nucleic acid in accordance with the ratio of magnetic beads detected in step (b) and step (f). This method advantageously allows to detect nucleic acids using CRISPR/Cas nucleases, which improves sensitivity, specificity allows for multiplexing, can be integrated in already existing platform technologies using f-TIR, such as the Atellica VTLi System from Siemens Healthineers.

In a preferred embodiment of the method the nucleic acid detector probe is composed of a capture probe being tethered to a substrate and a complementary binding probe segment, which is linked to a magnetic bead (binding molecule). In a further particularly preferred embodiment step (a) comprises the sub-steps (al) of providing a multitude of capture probes tethered to the substrate, and (a2) adding the binding molecule under conditions which allow for hybridization of the binding molecules to the capture probes.

In a further preferred embodiment, the capture probe and/or the binding molecule comprises a segment of locked nucleic acids (LNAs).

In yet another preferred embodiment said capture probe and said binding molecule comprise LNAs in a complementary segment.

According the present invention, it is further preferred that the nucleic acid detector probe is a single stranded DNA molecule or comprises a single stranded DNA portion, wherein said nucleic acid detector probe comprises a sequence segment which is complementary to the target nucleic acid and wherein the presence of the target nucleic acid being hybridized to the single stranded portion of the nucleic acid detector probe activates the nuclease complex and induces a specific cut of the hybridized nucleic acids. It is particularly preferred that the binding probe comprises a PAM sequence.

In another preferred embodiment of the method according to the present invention in step (a2) as described above the binding molecule is added together with a complex of a guidance nucleic acid and a not activated nuclease (nuclease complex) as well as a target nucleic acid, wherein said addition results in the provision of a nuclease activity, to induce degradation of the binding probe.

It is further preferred that the nucleic acid detector probe is a single stranded DNA or RNA molecule or comprises a single stranded portion, and that the presence of the target nucleic acid bound to the nuclease complex activates the nuclease complex and induces an unspecific collateral cut of the nucleic acid detector probe.

In yet another preferred embodiment, previous to step (d) as described above, the target nucleic acid is amplified. It is particularly preferred that said amplification is an isothermal amplification such as RT-LAMP, RPA or RT-RPA amplification.

The present invention further envisages in an embodiment that in steps (c) and/or (d) as described above the CRISPR-associated protein Csm6 or a functional equivalent thereof is added.

In an additional preferred embodiment, the nucleic acid detector probes on the substrate are directed to a multitude of different target nucleic acids. It is particularly preferred that said nucleic acid detector probes are directed to 2 to 30, preferably 2 to 18 different nucleic acid detector probes directed to the same target nucleic acid are provided as spots on the substrate.

In certain preferred embodiments of the method of the present invention, the nuclease is a Cas9 nuclease or a functional equivalent thereof. In other preferred embodiments of the method of the present invention the nuclease is a Cas12 nuclease or a Cas13 nuclease or a functional equivalent thereof.

In a further aspect that present invention relates to the use of nucleic acid detector probes and a nuclease complex as defined in a method according to the present invention for the detection of a target nucleic by frustrated total internal reflection (f-TIR) measurement.

### BRIEF DESCRIPTION OF THE DRAWINGS

FIG. 1 shows a schematic illustration of an embodiment of the invention. Depicted are magnetic beads (2) which are tethered to a substrate (1) with a nucleic acid detector probe (3). Upon activation (4) of a nuclease, e.g., of the type Cas12 or Cas 13, the magnetic beads (2) are removed from the substrate when the nucleic acid detector probe cut by the nuclease (5). The cleavage is a trans cleavage if a single stranded nucleic acid sequence is present in the vicinity of an active nuclease complex.

FIG. 2 shows another schematic illustration of an embodiment of the invention. Depicted are magnetic beads (2) which are tethered to a substrate (1) with nucleic acid detector probes as single stranded (3) or double stranded nucleic acid molecules (10). Upon activation (11) of a nuclease, e.g., of the type Cas9, the magnetic beads (2) are removed from the substrate when cut by the nuclease (12). The cleavage is a cis cleavage if a double stranded nucleic acid detector probe is present. Single stranded nucleic acid molecules (3) are not cleaved.

FIG. 3 depicts a further schematic illustration of an embodiment of the invention. On the left hand side, a substrate (1) with tethered capture probes (20) is shown. Binding probes bound to beads (22) (binding molecules) are flown in (21), which leads to the binding of the binding molecules (22) to the capture probes (20), being tethered to the substrate (1), in a complementary binding probe segment. Subsequently, samples and nucleases, e.g., of the type Cas 12 and Cas13 (24) are flown in (23). In case a target nucleic acid (25) is present in the sample the nuclease is activated and cleaves in trans (26) the single stranded portion of the complex of binding molecule (22) and capture probes (20). In case a target nucleic acid is absent from the sample, the nuclease is inactive and there is no cleavage (26) of the complex of binding molecule and capture probes.

FIG. 4 depicts a further schematic illustration of an embodiment of the invention. On the left-hand side, a substrate (1) with tethered capture probes (20) is shown. Binding molecules comprising beads (22) are flown in (21), which leads to the binding of the binding molecule (22) to the capture probes (20), being tethered to the substrate (1). Subsequently, samples with target nucleic acids (31) are flown in (30). The target nucleic acid (31) binds to the single stranded portion of the binding molecule (22). Then, nucleases, e.g., of the type Cas 9, (24) is flown in (32). In case a target nucleic acid (31) is present in the sample the nuclease is activated and cleaves (33) the double stranded portion of the complex. In case a target nucleic acid is absent from the sample, the nuclease is inactive and there is no cleavage (34).

FIG. 5 depicts another schematic illustration of an embodiment of the invention. On the left-hand side, a substrate (1) with tethered capture probes (20) is shown. Subsequently, a binding probe bound to a magnetic bead (binding molecule) (22) and a nuclease, e.g., of the type Cas 12 or Cas13, (24) and a target nucleic acid (41) are flown in (40). The presence of the target nucleic acid (41) activates the nuclease (42). In the presence of the target nucleic acid (41) the magnetic beads are removed from the rest of the binding molecule (43), whereas in the absence of the target nucleic acid no cleavage occurs (44). Subsequently, the mixture is provided to a substrate comprising capture probes. Consequently, in the presence of a target nucleic acid (41) in the sample the magnetic beads cannot be tethered to the substrate but remain in the reaction mixture (46) and are washed out. In case there is no target nucleic acid present, the magnetic beads become tethered to the substrate via the capture probes (47).

FIG. 6 depicts a further schematic c illustration of an embodiment of the invention. On the left-hand side, a substrate (1) with tethered capture probes (20) is shown. Subsequently a binding molecule with a magnetic bead and a nuclease, e.g., of the type Cas 9, (24) and sample are flown in (50). In case the sample comprises a target nucleic acid (51) the target nucleic acid (51) binds to the complementary single stranded segment of the binding molecule (22). The presence of the target nucleic acid (51) bound to the binding molecule then activates the nuclease (24). In the presence of the target nucleic acid the magnetic beads are removed from the rest of the binding molecule (52), whereas in the absence of the target nucleic acid no cleavage occurs, and the binding molecule (22) stays intact. Subsequently, the reaction mixture is provided to a substrate comprising capture probes. In the presence of a target nucleic acid in the sample the binding molecule remnants comprising magnetic beads (56) cannot be tethered but remain in the reaction mixture (54) and are subsequently washed out. In case there is no target nucleic acid present, the magnetic beads become tethered to the substrate via the capture probes (55).

FIG. 7 depicts an exemplary embodiment according to the present invention. On the left-hand side, a spot with a COVID-19 probe (60) is depicted, which is shown enlarged (61). The process starts with a substrate (1) to which COVID-19 capture probes (66) are tethered. Subsequently, magnetic beads bound to binding probes for FluA (62), FluB (63), RSV (64) and COVID-19 (65) (binding molecules) are flown in. Only the COVID-19 binding molecule (65) is able to bind the corresponding capture probe (66). Then, a sample with target nucleic acid (67) is flown in (68). The target nucleic acid (67) binds to the complementary single stranded segment of the COVID-19 binding molecule (65). Upon addition activation of nucleases, e.g., of the type Cas9, (24) a cleavage of the double stranded complex occurs (69) and binding molecule remnants with magnetic beads (73) are removed and can be washed out. Accordingly, a signal can be detected (72). In case an RSV binding molecule (64) binds to a corresponding capture probe (70) and further assuming no RSV target nucleic acid is present, no cleavage occurs, and no signal can be detected (71), as shown in the central lower part.

FIG. 8 shows an exemplary embodiment according to the present invention. Depicted are signals (88) and time (89). At a first time point (80) a cartridge is inserted, subsequently a reference readout is performed (81), then the binding probe is added (82). At a next time point (83) a washing step occurs, followed by read-out 1 (84), the addition of a sample comprising the target nucleic acid (85), the addition of a nuclease (e.g. Cas12 and activating ingredients) (86) and the performance of a further washing step (83). Finally, a read-out 2 step is performed (87). The signal difference between read-out 1 and read-out 2 is shown (90).

FIG. 9 shows another exemplary embodiment according to the present invention. Depicted are signals (88) and time (89). At a first time point (80) a cartridge is inserted, subsequently a sample is added, wherein the sample comprises a target nucleic acid premixed with a binding probe and a Cas12 complex (93). Read-out curves in the absence of a target nucleic acid (92) and in the presence of a target nucleic acid (91) are shown. Further depicted are the differences in read-out signals with respect to a control signal in the absence of a targe nucleic acid (94) and in the presence of a target nucleic acid (95).

### DETAILED DESCRIPTION OF EMBODIMENTS

Although the present invention will be described with respect to particular embodiments, this description is not to be construed in a limiting sense.

Before describing in detail exemplary embodiments of the present invention, definitions important for understanding the present invention are given.

As used in this specification and in the appended claims, the singular forms of “a” and “an” also include the respective plurals unless the context clearly dictates otherwise.

In the context of the present invention, the terms “about” and “approximately” denote an interval of accuracy that a person skilled in the art will understand to still ensure the technical effect of the feature in question. The term typically indicates a deviation from the indicated numerical value of +20%, preferably +15%, more preferably +10%, and even more preferably +5%.

It is to be understood that the term “comprising” is not limiting. For the purposes of the present invention the term “consisting of” or “essentially consisting of” is considered to be a preferred embodiment of the term “comprising of”. If hereinafter a group is defined to comprise at least a certain number of embodiments, this is meant to also encompass a group which preferably consists of these embodiments only.

Furthermore, the terms “(i)”, “(ii)”, “(iii)” or “(a)”, “(b)”, “(c)”, “(d)”, or “first”, “second”, “third” etc. and the like in the description or in the claims, are used for distinguishing between similar or structural elements and not necessarily for describing a sequential or chronological order.

It is to be understood that the terms so used are interchangeable under appropriate circumstances and that the embodiments of the invention described herein are capable of operation in other sequences than described or illustrated herein. In case the terms relate to steps of a method, procedure or use there is no time or time interval coherence between the steps, i.e., the steps may be carried out simultaneously or there may be time intervals of seconds, minutes, hours, days, weeks etc. between such steps, unless otherwise indicated.

It is to be understood that this invention is not limited to the particular methodology, protocols etc. described herein as these may vary. It is also to be understood that the terminology used herein is for the purpose of describing particular embodiments only and is not intended to limit the scope of the present invention that will be limited only by the appended claims.

The drawings are to be regarded as being schematic representations and elements illustrated in the drawings are not necessarily shown to scale. Rather, the various elements are represented such that their function and general purpose become apparent to a person skilled in the art.

Unless defined otherwise, all technical and scientific terms used herein have the same meanings as commonly understood by one of ordinary skill in the art.

Independent of the grammatical term usage, individuals with male, female or other gender identities are included within the term.

As has been set out above, the present invention concerns in one aspect a method for the detection of a target nucleic acid, comprising the steps: (a) providing a multitude of nucleic acid detector probes, each being linked to a magnetic bead and tethered to a substrate; (b) detecting the number of magnetic beads linked to the substrate by frustrated total internal reflection (f-TIR); (c) providing a complex of a guidance nucleic acid and a not activated nuclease (nuclease complex); (d) activating the nuclease complex of step (c) by the addition of a target nucleic acid, wherein said activation results in the provision of a nuclease activity, to induce degradation of the nucleic acid detector probe; (e) removing unbound magnetic beads; (f) detecting the number of magnetic beads linked to the substrate by f-TIR; and (g) determining the amount of target nucleic acid in accordance with the ratio of magnetic beads detected in step (b) and step (f).

The term “nucleic acid detector probe” as used herein refers to a nucleic acid molecule, which is single stranded. The nucleic acid molecule may have any suitable form or structure and should be cleavable by a nuclease, preferably by a Cas type nuclease. In one embodiment, the nucleic acid is a DNA molecule, e.g., a single stranded DNA molecule. In a further embodiment, the nucleic acid may be an RNA molecule. In further embodiments, the nucleic acid may comprise PNA, CNA, HNA, LNA or ANA segments or mixtures thereof. The term “PNA” as used herein relates to a peptide nucleic acid, i.e., an artificially synthesized polymer similar to DNA or RNA. The PNA backbone is typically composed of repeating N-(2 -aminoethyl)-glycine units linked by peptide bonds. The various purine and pyrimidine bases are linked to the backbone by methylene carbonyl bonds. The term “CNA” as used herein relates to a cyclopentane nucleic acid, i.e., a nucleic acid molecule comprising for example 2′-deoxycarbaguanosine. The term “HNA” relates to hexitol nucleic acids, i.e., DNA analogues which are built up from standard nucleobases and a phosphorylated 1, 5-anhydrohexitol backbone. The term “LNA” as used herein relates to locked nucleic acids. Typically, a locked nucleic acid is a modified and thus inaccessible RNA nucleotide. The ribose moiety of an LNA nucleotide may be modified with an extra bridge connecting the 2′ and 4′ carbons. Such a bridge locks the ribose in a 3′-endo structural conformation. The locked ribose conformation enhances base stacking and backbone pre-organization. This is assumed to increase the thermal stability, i.e., melting temperature of the oligonucleotide. The term “ANA” as used herein relates to arabinoic nucleic acids or derivatives thereof. A preferred ANA derivative in the context of the present invention is a 2′-deoxy-2′-fluoro-beta-D-arabinonucleoside (2′F-ANA).

The nucleic acid detector probe may have any suitable length, which may be adapted to the method, environment, device, reaction to be performed, target nucleic acid etc. In typical embodiments, the nucleic acid detector probe has a length of 40 to 250 nucleotides, e.g., 40, 45, 50, 55, 60, 65, 100, 150, 200 or 250 nucleotides or any value in between the mentioned values. It is preferred that the length of the oligonucleotide is 50-100 nucleotides.

The “magnetic bead” to which the detector probe is linked may have any suitable form. Typically, the magnetic beads may include iron oxide such as Fe₃O₄, or Fe₂O₃, or iron platinum.

Also envisaged are alloys with Ni, Co and Cu, or particles comprising these elements. In certain embodiments, the magnetic bead may comprise a certain amount of superparamagnetic material, e.g., 10%, 20%, 30%, 40%, 50%, 60%, 70%, 80%, 90% or more than 90% by weight. The beads may, for example, comprise an encapsulation with a polymer coating such as polystyrene. In preferred embodiments, the material comprised in the magnetic particle may have a saturated moment per volume as high as possible thus allowing to maximize gradient related forces.

The magnetic beads may have any suitable form, e.g., be of a symmetrical, globular, essentially globular or spherical shape, or be of an irregular, asymmetric shape or form. The size of a magnetic bead envisaged by the present invention may range between 50 nm and 1000 nm. Preferred are magnetic beads in the nanometer range. In preferred embodiments the magnetic bead diameter is larger than 100 nm. Particularly preferred are magnetic beads of a size of 300 nm to 1000 nm, e.g., 300 nm, 400 nm, 500 nm, 600 nm, 700 nm 800 nm, 900 nm, or 1000 nm, or any value in between. Even more preferred are magnetic beads having a diameter of about 500 nm.

Particularly preferred are magnetic beads which are polystyrene spheres filled with small magnetic grains (e. g. of iron-oxide as described above), rendering the beads superparamagnetic.

The term “substrate” as used herein refers to a solid phase present at the surface of an entity, which is typically composed of porous and/or non-porous material, usually insoluble in water. The substrate may have various forms such as a vessel, tube, microtitration plate, or cartridge etc. Usually, the surface of the solid phase is hydrophilic. The substrate may be composed of various materials such as materials and/or organic materials, inorganic synthetic materials, naturally occurring materials and/or modified naturally occurring materials. Examples of substrate materials include polymers such as cellulose, nitrocellulose, cellulose acetate, polyvinyl chloride, polyacrylamide, cross-linked dextran molecules, agarose, polystyrene, polyethylene, polypropylene, polymethacrylate, or nylon; ceramics; silicate; glass; metals, e.g., noble metals such as gold and silver; or mixtures or combinations thereof. In preferred embodiments, the substrate is composed of polystyrene. It is particularly preferred that refractive index of the polystyrene matches with the refractive index of the polystyrene coated magnetic beads, which allows for optical outcoupling of light in a f-TIR analysis.

Within the context of f-TIR analysis the substrate as defined above is preferably designed as a sensor surface. The term “sensor surface” refers to a flat surface which is capable of generally interacting with the magnetic bead, e.g., by tethering or linking them. The sensor surface may accordingly be functionalized with interactive entities or other functional elements, e.g., chemical groups, allowing to link the surface to a nucleic acid. The sensor surface may further be connected with downstream electronic or optical or magnetic etc. devices allowing to perform additional activities on the magnetic bead.

The term “multitude of nucleic acid detector probes” as used herein refers to an amount of nucleic acid molecules which is adapted to and optimized for the detection procedure, in particular the f-TIR detection, as well as the enzymatic activity of the nuclease and the expected amount of target nucleic acid in a sample. In typical embodiments an amount of 10⁷ to 10¹², preferably 10¹⁰ nucleic acid detector probes may be provided per mm² of the substrate.

In a subsequent step the number of magnetic beads linked to the substrate is detected by f-TIR. The term “f-TIR” stands for “frustrated total internal reflection”. As used herein a “total internal reflection” describes a condition present in certain materials when light enters one material from another material with a higher refractive index at an angle of incidence greater than a specific angle. The specific angle at which this occurs depends on the refractive indices of both materials, also referred to as critical angle and can be calculated mathematically (Snell's law, law of refraction). In absence of particles, e.g., magnetic beads, no refraction occurs and the light beam from the light source is totally reflected. If a particle, e.g., magnetic bead, is close to the sensor surface or is in contact with the sensor surface the light rays are said to be “frustrated” by the magnetic bead and reflection at that point is no longer total. The signal, which may be defined as the decrease of the totally internal reflected signal can be calculated. This signal is more or less linearly dependent on the concentration or number of particles, e.g., magnetic beads, on the surface (surface density ñ).

The signal can be expressed as (Formula 1):

$$S = β \tilde{n} \qquad ( 1 )$$

wherein S is the measured signal change in % and β is a conversion factor from surface density to signal change. The detection of bound particles, e.g. magnetic beads, thus occurs via f-TIR. In particular, f-TIR detection is based on the measurement of the reflected light intensity from the surface where the particles, e.g. magnetic beads, are bound. Depending on the amount of particles, e.g. magnetic beads, at the surface, light is scattered from the light beam or absorbed, causing a reduction in the reflected light intensity. By comparing the reflected light intensity at different time points (e.g. before, after, or at multiple stages during the assay), the signal change can be determined. Preferably, the signal change is compared to a master-curve to convert signal change in e.g. an amount of bound particles, e.g. magnetic beads, or an analyte concentration.

The detection may, in preferred embodiments, be carried out in an optomagnetic system, wherein said magnetic beads are firstly magnetically actuated, e.g. non-bound magnetic particles can be removed via magnetic force, i.e. magnetic beads-magnetic force interaction. Subsequently, the magnetic beads still present at the sensor surface may then be detected optically, e.g. within in a stationary reaction mixture covering the sensor surface, comprising, inter alia, a sample to be measured. In certain embodiments the washing may be repeated, e.g., the washing may be performed twice or more often. Preferred is a washing performed twice.

The present invention envisages, in certain embodiments, also the method to be implemented in a different format, e.g. with fluorescence imaging detection. Accordingly, the detection may be performed with fluorescence or chemiluminescence labels attached to the detector probes or binding probes. Thus, in these embodiments, instead of a magnetic bead a fluorescent or chemiluminescence label is linked to the detector probe or binding probe. In further embodiments, the method may alternatively be performed on substrate present on beads as described herein. For example, a fluorescently labeled detector probe or binding probe on a bead may be detected with fluorescent flow cytometric read out. In these examples, a washing step, preferably with buffer, is typically performed in order to increase the sensitivity.

In preferred embodiments, the optomagnetic system may be an f-TIR system known to the skilled person such as the Atelic VTLi System. Typically, the f-TIR system is provided as handheld device which can be equipped with cartridges comprising all ingredients for performing the method of the present invention, e.g. offering an entry spot for a sample and ad display for a result or an interface for cloud or server connectivity.

The accordingly measured number of magnetic beads is to be seen as starting or reference number which can be compared with subsequently determined numbers of magnetic beads, in particular, after the detection of target nucleic acids has been performed.

In a further step of the method according to the present invention a complex of a guidance nucleic acid and a not activated nuclease, i.e., a nuclease complex is provided. This complex may, for example, be introduced into a reaction zone, reaction chamber, vessel or any other suitable room which surrounds the substrate, i.e., the sensor surface. It is particularly preferred that said reaction zone is cartridge, e.g., a cartridge which can be introduced into a f-TIR device such as the Atellica VTLi System.

The method according to the present invention is, in general, based on the employment of nucleases which belong to the CRISPR/Cas system, or any equivalent system known to the skilled person or to be developed in the future. The term “CRISPR/Cas system” as used herein relates to a biochemical method to specifically cut and modify nucleic acids, also known as genome editing. For example, genes in a genome can generally be inserted, removed or switched off with the CRISPR/Cas system, nucleotides in a gene or nucleic acid molecule can also be changed. The effect of the concept and activity steps of the CRISPR/Cas system has various similarities to that of RNA interference since short RNA fragments of about 18 to 20 nucleotides mediate the binding to the target in both bacterial defense mechanisms. In the CRISPR/Cas system typically RNA-guided nucleic acid-binding proteins, such as Cas proteins, bind certain RNA sequences as ribonucleoproteins. For example, a Cas endonuclease (e.g., Cas9, Cas5, Csn1 or Csx12, or derivatives thereof) can bind to certain RNA sequences termed crRNA repeats and cut DNA in the immediate vicinity of these sequences. Without wishing to be bound by theory, it is believed that the crRNA repeat sequence forms a secondary RNA structure and is then bound by the nuclease (e.g., Cas) which alters its protein folding allowing the target nucleic acid to be bound by the RNA. Furthermore, the presence of a PAM motif, i.e., a protospacer adjacent motif, in the target DNA is necessary to activate the nuclease (e.g., Cas). The DNA is typically cut three nucleotides before the PAM motif. The crRNA repeat sequence is typically followed by a sequence binding to the target DNA, i.e., a crRNA spacer; both sequences, i.e., the crRNA repeat motif and the target binding segment are usually labelled as “crRNA”. This second part of the crRNA (target binding segment) is a crRNA-spacer sequence having the function of a variable adapter. It is complementary to the target DNA and binds to said target DNA. An additional RNA, a tracrRNA, or trans-acting CRISPR RNA, is also required. tracrRNA is partially complementary to crRNA, so that they bind to each other. tracrRNA typically binds to a precursor crRNA, forms an RNA double helix and is converted into the active form by RNase III. These properties allow for a binding to the DNA and a cutting via the endonuclease function of the nuclease (e.g., Cas) near the binding site.

In this context the term “guidance nucleic acid” as used herein refers to nucleic acid sequences which are bound by the nucleases, in particular Cas nucleases, and activate their cleavage functionality It is preferred that these guidance nucleic acids are artificially produced and thus tailored for the specifically intended usage. An example of such artificially generated guidance nucleic acids is “single guide RNAS (sgRNAs)”, i.e., an artificial or synthetic combination of a crRNA and a tracrRNA sequence of the CRISPR/Cas system as described above. Typically, the sgRNA comprises a target specific sequence which can be used to guide a DNA binding protein towards the binding site. As described in Jinek et al., 2012, Science, 337, 816-821 crRNA and tracrRNA can be combined into a functional species (sgRNA) which fulfills both activities (crRNA and tracrRNA) as mentioned above. For example, nucleotides 1-42 of crRNA-sp2, nucleotides 1-36 of crRNA-sp2 or nucleotides 1-32 of crRNA-sp2 may be combined with nucleotides 4-89 of tracrRNA. Further options for obtaining an sgRNA can be derived from Nowak et al., 2016, Nucleic Acids Research, 44, 20, 9555-9564. For example, an sgRNA may be provided which comprises different forms of an upper stem structure, or in which the spacer sequence is differentially truncated from a canonical 20 nucleotides to 14 or 15 nucleotides. Further envisaged variants include those in which a putative RNAP III terminator sequence is removed from the lower stem. Also envisaged is a variant, in which the upper stem is extended to increase sgRNA stability and enhance its assembly with an sgRNA-guided nuclease, e.g., Cas protein. According to further embodiments of the present invention, the sequence and form of the sgRNA may vary in accordance with the form or identity of the sgRNA-guided nuclease, e.g., the different Cas proteins used. Accordingly, depending on the original of said sgRNA-guided nucleic acid-binding protein, a different combination of sequence elements may be used. The present invention further envisages any future development in this context and includes any modification or improvement of the sgRNA-nucleic acid-binding protein interaction surpassing the information derivable from Jinke et al., 2012 or Nowak et al., 2016. Particularly is the of preferred use a Streptococcus pyogenes sgRNA, e.g., as used in commercially available kits such as EnGen sgRNA synthesis Kit provided by New England Biolabs Inc. Also envisaged are similar sgRNA forms from other commercial suppliers, or individually prepared sgRNAs.

The features necessary to prepare a synthetic single guide RNA (sgRNA), in general, comprise all elements which are necessary to generate an sgRNA molecule suitable for employment in a CRISPR/Cas system as described herein above. Accordingly, these features include the presence of a promoter segment; the presence of a random segment as target specific sequence which serves as complementary sequence for a potential binding or hybridization interactor having a matching sequence; and the presence of a binding element which is complementary to at least a portion of a scaffold sequence for interaction with the sgRNA-guided nucleic acid-binding protein. The mentioned features may be provided in any suitable order.

Accordingly, a “nuclease complex” as mentioned herein comprises a nuclease, e.g., a Cas protein, and a suitable, i.e., compatible crRNA or sgRNA. In the absence of a target nucleic acid this complex is non-working and can be hold available for a certain time, e.g., until a sample or target nucleic acid is added. For example, the nuclease complex or components therefore may be provided in a lyophilized form, e.g., as lyophilized reaction mix, which can be dissolved upon starting of the method or when introducing a liquid sample or the like.

As outlined above, the activation of the nuclease, e.g., Cas, implies binding of a nucleic acid structure and a target nucleic acid. This target nucleic acid may, for example, be part of a double-strand DNA molecule, a single-strand DNA molecule or an RNA molecule. Depending on the nature and form of the target nucleic acid to be detected a different nuclease or set of nucleases, i.e., a different type of Cas nuclease and concomitant assay format may be used.

The target nucleic acid may, in typical embodiments, be a nucleic acid present in a sample. It may be nucleic acid, which is derived from a virus or bacterium or any other organism, or any other nucleic acid of interest including artificial nucleic acids. It is preferred that the target nucleic acid is a viral or bacterial nucleic acid, e.g., typical for a viral or bacterial infection. Particularly preferred are target nucleic acids from a DNA or RNA virus, e.g. dsDNA virus such as a virus belonging to Caudovirales, Herpesvirales or Ligamenvirales, the family of Adenoviridae, Ampullaviridae, Ascoviridae, Asfarviridae, Baculoviridae, Bicaudaviridae, Clavaviridae, Corticoviridae, Fuselloviridae, Globuloviridae, Guttaviridae, Hytrosaviridae, Iridoviridae, Lavidaviridae, Marseilleviridae, Mimiviridae, Nimaviridae, Nudiviridae, Pandoraviridae, Papillomaviridae, Phycodnaviridae, Plasmaviridae, Polydnaviruses, Polyomaviridae, Poxviridae, Sphaerolipoviridae, Tectiviridae, Tristromaviridae or Turriviridae. It may also be a ssDNA virus such as a virus belonging to the family of Anelloviridae, Bacilladnaviridae, Bidnaviridae, Circoviridae, Geminiviridae, Genomoviridae, Inoviridae, Microviridae, Nanoviridae, Parvoviridae, Smacoviridae or Spiraviridae. The virus may further be a dsDNA virus belonging to the family of Amalgaviridae, Birnaviridae, Chrysoviridae, Cystoviridae, Endornaviridae, Hypoviridae, Megabirnaviridae, Partitiviridae, Picobirnaviridae, Reoviridae, Totiviridae, Quadriviridae, or Botybirnavirus. It may further be negative strand SSRNA virus, e.g., belonging to the order of Muvirales, Serpentovirales, Jingchuvirales, Mononegavirales, Goujianvirales, Bunyavirales or Articulavirales, or Filoviridae, Paramyxoviridae, Pneumoviridae or Orthomyxoviridae. In particular, it may be a RSV, metapneumovirus, or an influenza virus. The virus may further be a positive strand ssRNA virus such as a virus belonging to the order of Nidovirales, Picornavirales or Tymovirales, in particular a virus belonging to the family of Coronaviridae, Picornaviridae, Caliciviridae, Flaviviridae or Togaviridae. It is further preferred that the virus is a rhinovirus, Norwalk-Virus, Echo-Virus or enterovirus. In a particularly preferred embodiment said virus is PHEV, FcoV, IBV, HCoV-OC43, HcoV HKU1, JHMV, HCOV NL63, HCOV 229E, TGEV, PEDV, FIPV, CCOV, MHV, BCOV, SARS-COV, MERS-COV or SARS-COV-2 virus, or any mutational derivative thereof. Further preferred are target nucleic acids from a bacterium which causes a human infection such as ESKAPE bacteria (Enterococcus faecium, Staphylococcus aureus, Klebsiella pneumoniae, Acinetobacter baumannii, Pseudomonas aeruginosa and Enterobacter species), Neisseria or Chlamydia species, or any other bacterium of interest as known to the skilled person.

The target nucleic acid may be added to the nuclease complex as described above in any suitable form. For example, the nucleic acid may be flown into a reaction zone or chamber or vessel comprising the tethered magnetic beads as test sample. The term “test sample” as used herein relates to any biological material obtained via suitable methods known to the person skilled in the art from a subject. The sample used in the context of the present invention should preferably be collected in a clinically acceptable manner, more preferably in a way that nucleic acids are preserved. The biological samples may include body tissues and/or fluids, such as blood, or blood components like serum or plasma, sweat, sputum or saliva, semen and urine, as well as feces or stool samples. Furthermore, the biological sample may contain a cell extract derived from or a cell population including an epithelial cell. In certain embodiments cells may be used as primary sources for polynucleotides. Accordingly, the cells may be purified from obtained body tissues and fluids if necessary, and then further processed to obtain polynucleotides. In certain embodiments samples, in particular after initial processing, may be pooled. The present invention preferably envisages the use of non-pooled samples. In a specific embodiment of the present invention the content of a biological sample may also be submitted to a specific pre-enrichment step. In further embodiments of the invention, biopsy or resections samples may be obtained and/or used. Such samples may comprise cells or cell lysates. Furthermore, cells may be enriched via filtration processes of fluid or liquid samples, e.g., blood, urine, sweat etc. Such filtration processes may also be combined with pre-enrichment steps based on ligand specific interactions as described herein above.

In preferred embodiments, the sample flown in reaction zone may comprise purified nucleic acids. The sample can further be processed or be partially processed, e.g., by heating, treatment, by purifying it with ion exchange material such as chelex-100 or by any other methods. Further, the sample may be diluted or condensed, depending on its concentration and the envisaged usage.

In certain situations, the concentration or amount of target nucleic acid in the test sample may be low, thus interfering with the performance of the detection steps as outlined herein. Therefore, in specific embodiments, the present invention envisages an amplification procedure for said target nucleic acids. The amplification may take place in the reaction zone, thus requiring suitable conditions which do not compromise the nuclease-based detection steps. Alternatively, the amplification may be performed outside of the reaction zone, e.g., after preparation of nucleic acids from a sample. For such an outside amplification typically a PCR method may be employed, e.g., a standard PCR as known to the skilled person. Alternatively, asymmetrical PCR, i.e., a method to preferentially amplify one strand of the original DNA more than other, the may be performed. Further information on suitable PCR techniques would be known to the skilled person or can be derived from suitable literature sources such as Domingues ed., 2017, PCR Methods and Protocols, in Methods in Molecular Biology 1620, Springer Protocols, Humana Press.

Preferred amplification methods inside of the reaction zone or cartridge are isothermal amplification methods. These methods amplify a nucleic acid in a streamlined, exponential manner and are not limited by the constraint of thermal cycling. In particular, the nucleic acid strands are not heat denatured a polymerase with strand-displacement activity is required. Examples of isothermal amplification envisaged by the present invention include Loop-mediated isothermal amplification (LAMP). This method typically uses 4-6 primers recognizing 6-8 distinct regions of a target nucleic acid for a highly specific amplification reaction. A strand-displacing DNA polymerase initiates synthesis and 2 specially designed primers form loop structures to facilitate subsequent rounds of amplification through extension on the loops and additional annealing of primers. Long DNA products are formed as concatemers. A further envisaged example of an isothermal amplification method is strand displacement amplification (SDA), which relies on a strand displacement DNA polymerase and a DNA nicking event targeted via primer design and a nicking endonuclease. The nicking site is regenerated with each polymerase displacement step for repeated cycles of nicking and extension, with the downstream strand displaced and free to anneal to primers in solution for amplification from the other end, resulting in exponential amplification. The nature of the SDA reaction produces discrete fragments of DNA. A further example is Helicase-dependent amplification (HDA), which employs the double-stranded DNA unwinding activity of a helicase to separate strands, enabling primer annealing and extension by a strand-displacing DNA polymerase. Also envisaged is recombinase polymerase amplification (RPA) and strand-invasion based amplification (SIBA) which make use of the activity of a recombinase enzyme that help primers invade into double-stranded DNA. Further envisaged are Nucleic Acid Sequenced Based Amplification (NASBA) and Transcription Mediated Amplification (TMA) which proceed through RNA. Typically, primers are designed to target a region of interest. One primer includes a promoter sequence for T7 RNA polymerase at the 5′ end. This enables production of single-stranded RNA species, which are reverse transcribed to cDNA by a reverse transcriptase included in the reaction. The RNA in the DNA-RNA hybrids is destroyed by RNase H activity and dsDNA is produced by the RT. Also, a rolling circle amplification (RCA) is envisaged, wherein a DNA polynucleotide, which is typically short, is amplified to form a long single stranded DNA polynucleotide using a circular DNA template and a suitable polymerase. The RCA product is typically a concatemer containing several, e.g., 5 to 500 tandem repeats that are complementary to the circular template. The methods may further be combined with reverse transcriptase (RT) steps in order to amplify RNA molecules. Preferred is the employment of RT-LAMP, RPA or RT-RPA. Further details would be known to the skilled person or can be derived from suitable literature sources such as Thompson and Lei, 2020, Sensors and Actuators Report, 2, 1, 100017; or Sun et al., 2021, J. Trans. Med., 19, 74.

In further specific embodiments the generated nucleic acids, which are typical DNA molecules, may additionally be subjected to a transcription step to produce an RNA molecule. Such an RNA target nucleic acid may advantageously be used in the context of a method which employs RNA specific nucleases, i.e., Cas proteins such as Cas13.

The activation of the nuclease complex, e.g., Cas complex, results in the provision of a nuclease activity to induce degradation of the nucleic acid detector probe. The degradation and its mechanics largely depend on the nuclease used, in particular on the class of Cas nucleases employed. The present invention envisages the use of all suitable Cas classes, which are capable cleaving DNA and RNA molecules. It is particularly preferred to use Cas 9, Cas12 and Cas13 nucleases, as well as derivatives or mutated sub-forms or functional equivalents thereof.

Cas9 belong to the type II CRISPR/Cas system. It is derived from Streptococcus pyogenes, Streptococcus thermophilus or other bacteria. Typically, under natural processes, the trans-activating crRNA (tracrRNA) base pairs with the repeat sequence in the crRNA to form a unique dual RNA hybrid structure guide that directs Cas9 to cleave the target DNA. sgRNA, as mentioned above, is designed to combine CRNA and tracrRNA and preserves Cas9's activity. Cas9 contains two nuclease domains, RuvC and HNH which cut the target DNA strands and non-target DNA strands respectively, i.e., make a blunt double-stranded DNA break. A short trinucleotide protospacer adjacent motif (PAM) is essential for initial target sequence recognition since the target sequence cannot be recognized without the PAM site. After successful identification, a double-strand break (DSB) occurs upstream of the 3′-NGG PAM site. Cas9 can hence preferably be used for the degradation of double-stranded DNA molecules in cis, e.g. double-stranded nucleic acid detector probes, since it has no collateral strand cleavage activity.

Cas12 belongs to the type V CRISPR/Cas system. It is derived from Francisella novicida, Acidaminococcus or other bacteria.

Cas12a, a sub-type, contains a predicted RuvC-like endonuclease domain, which can cleave dsDNA under the guidance of gRNA. Unlike Cas9, Cas12a recognizes a distal 5′-T-rich PAM and generates PAM distal dsDNA breaks with staggered 5′ and 3′ ends. Cas12a also can recognize complementary ssDNA sequences in a PAM-independent manner and cleave it. Different from Cas9, Cas12 has collateral strand cleavage activity as the target DNA sequence is present. Cas12a can hence preferably be used for the degradation of single-stranded DNA molecules in trans, e.g., single-stranded nucleic acid detector probes, since it has a trans or collateral strand cleavage activity.

Cas12b, another Cas12 sub-type, has the same non-specific trans-cleavage capability as Cas12a. However, the Cas12b system shows different target preferences during the trans cleavage process, in particular the non-specific ssDNA trans-cleavage rate is higher when dsDNA is used as the target than SSDNA. In addition, Cas 12b targets SSDNA substrates by cleaving the ssDNA probe independently of the PAM, whereas targeting dsDNA requires the 5′-TTN-3′ PAM site. Cas12b can hence also preferably be used for the degradation of single-stranded DNA molecules in trans, e.g., single-stranded nucleic acid detector probes, since it has a trans or collateral strand cleavage activity.

Cas13 belong to the type VI CRISPR/Cas system. It is derived from Leptotrichia buccalis, Leptotrichia shahii, Ruminococcus flavefaciens or other bacteria. Cas13 contains two nucleotide sequence binding domains (HEPN) and has single-stranded RNA (SSRNA) cleavage activity. Cas13 has collateral strand cleavage activity as the target RNA sequence is present. Thus, once it is activated by a ssRNA sequence bearing complementarity to its crRNA spacer, a nonspecific RNase activity is initiated which degrade all nearby RNA molecules regardless of their sequence. Cas13 can preferably be used for the degradation of RNA molecules in trans, e.g., single-stranded RNA detector probes, since it has a trans or collateral strand cleavage activity.

Further envisaged is, in specific embodiments, the use of additional Cas nucleases such as Cas14, which detects single DNA strands and cleaves ssDNA molecules. Further details on the form and use of Cas proteins may be derived from suitable literature sources such as Jiang and Doudna, 2017, Annu. Rev. Biophys., 46, 505-529, Makarova et al., 2011, Biology Direct, 6, 38 or Wang et al., 2016, Annu. Rev. Biochem., 85, 22.1-22.38.

Thus, in the presence of a corresponding target nucleic acid in a test sample, the nucleic acid detector probes may be cut and thus linked magnetic beads may be released. These unbound beads are subsequently removed from the reaction mixture, e.g., in the cartridge, with any suitable technique. For example, the method envisages washing or removal steps between. These removal steps may be implemented with any suitable technique, e.g., by removing or relocating the reactants, in particular removing the magnetic beads. The washing step may be performed once, or it may be repeated 1, 2, 3 or more times. It is preferred to perform the washing with nuclease-free water or with any other suitable solution containing appropriate ion concentration and/or having a suitable pH, as would be known to the skilled person.

In preferred embodiments, one or more of the removal or washing steps as mentioned herein above may be carried out with the help of a magnet capable of collecting magnetic beads. The beads may subsequently be washed with a continuous water flow, followed by the creation of vacuum which removes the water. It is particularly preferred that the washing and removal steps are performed in accordance with procedures described for the Atellica VTLi or Centaur assay platforms of Siemens Healthineers as described herein above or known to the skilled person.

Subsequent to the removal of the unbound magnetic beads a further detection of the number of magnetic beads linked to the substrate, i.e., on the surface of the device is performed, essentially as described herein, with the f-TIR approaches. The second measurement will detect only those magnetic beads which have not been rendered unbound by the cleavage of a nuclease, e.g. by the nuclease Cas. The amount of these magnetic beads can be determined with f-TIR.

The resulting amount is correlated to the previously determined number of magnetic beads, i.e., the starting number before the nuclease activity was initiated with the flowing in of the sample

Finally, the amount of target nucleic acids in sample is determined in accordance with the calculation of a ratio of magnetic beads detected in step (b) and step (f). Less detected magnetic beads indicate a higher concentration of a target nucleic acid in the tested sample. Thus, the difference of the number of magnetic beads determined before the activation of the nuclease and afterwards is proportional to the bound probe oligos which is further proportional to the concentration of target nucleic acid present. This difference can be translated into an indication of the concentration or amount of the target nucleic acid in the sample. In certain embodiments, suitable calibration procedures may be used to calibrate and adjust this process.

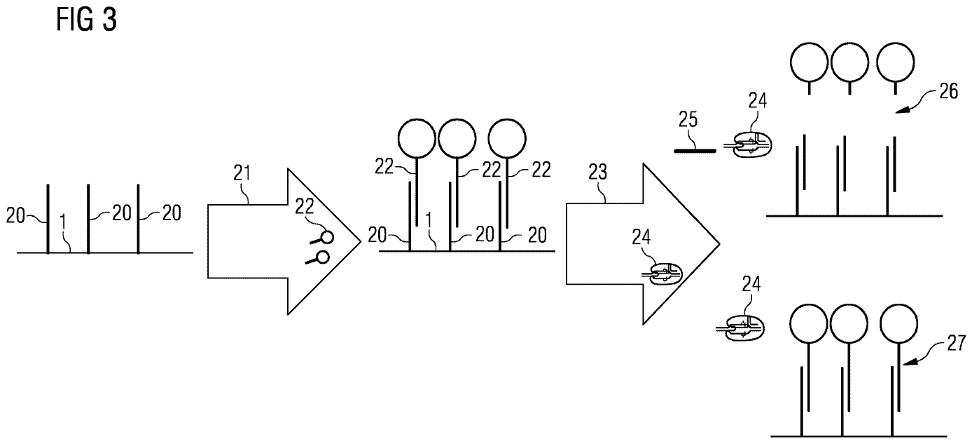

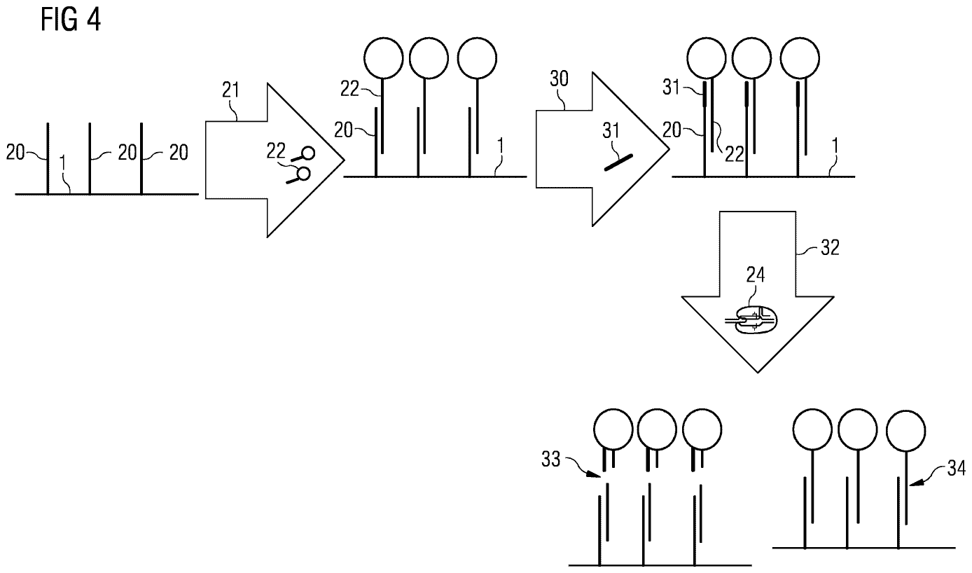

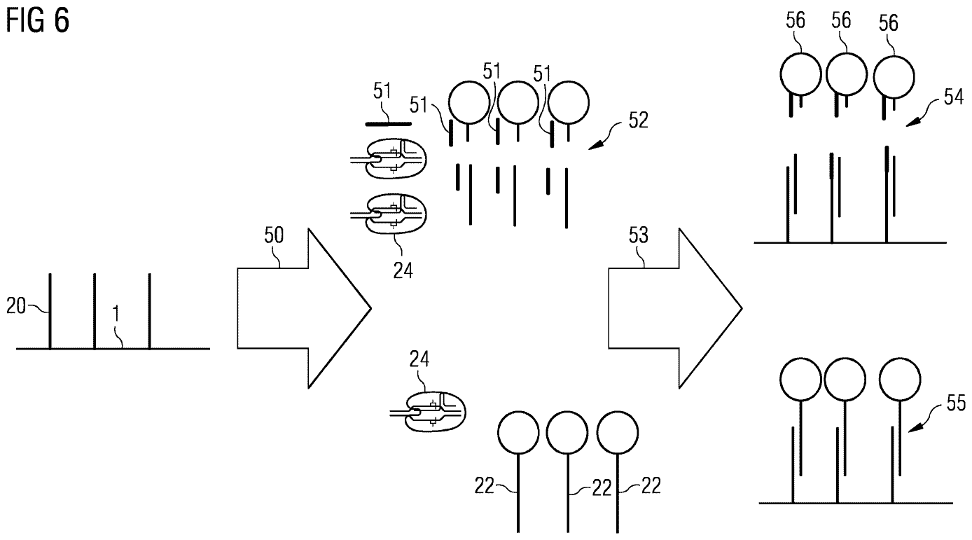

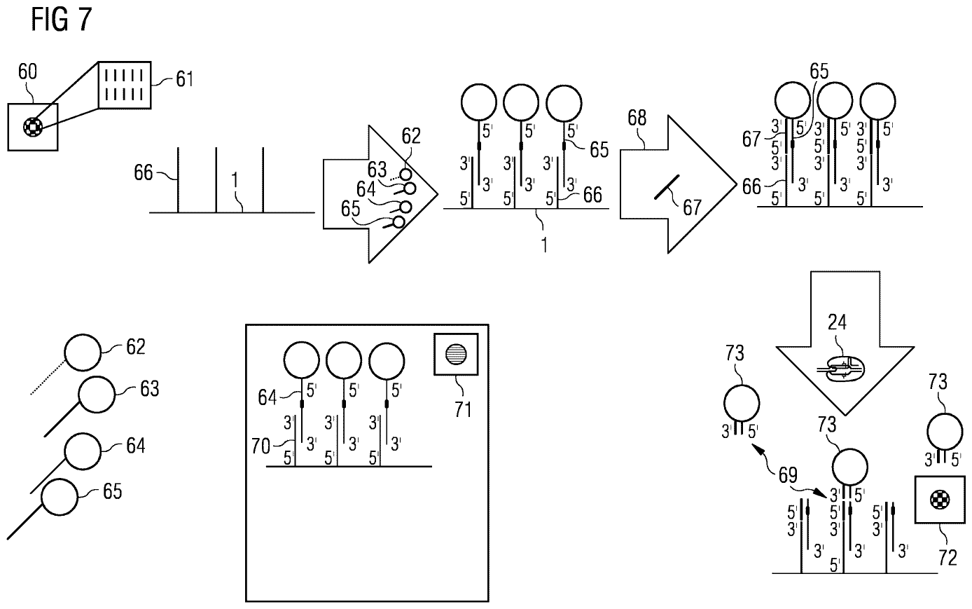

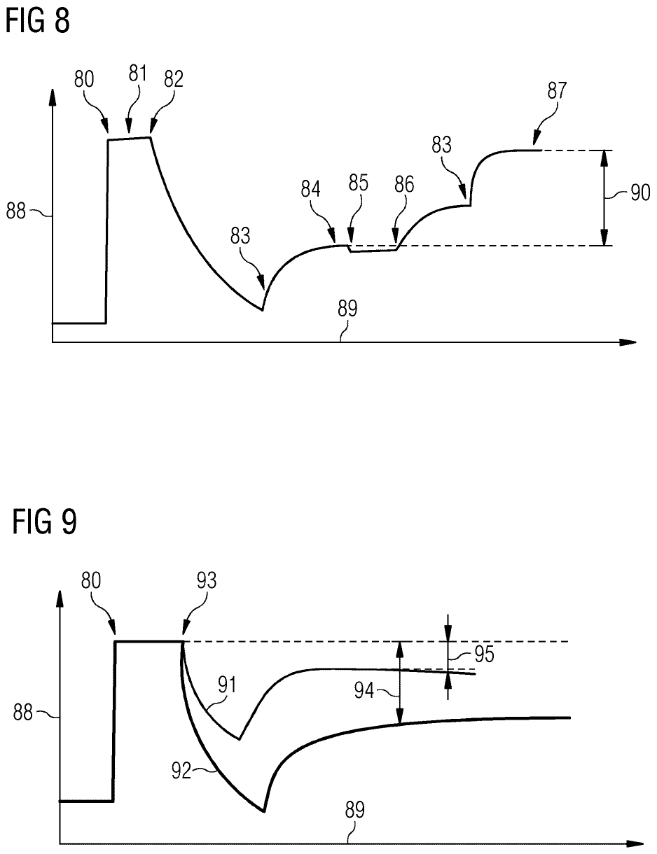

In a specific embodiment, the nucleic acid detector probe as described herein is composed of a capture probe being tethered to a substrate and a complementary binding probe segment, which is linked to a magnetic bead (binding molecule). Accordingly, the nucleic acid detector probe is composed two entities with an overlapping segment (see also FIGS. 3 to 6). The term “complementary” as used herein refers to the presence of matching base pairs in opposite nucleic acid strands. For example, to a nucleotide or base A in a sense strand a complementary or antisense strand binds with a nucleotide or base T, or vice versa; likewise to a nucleotide or base G in a sense strand the complementary or antisense strand binds with a nucleotide or base C, or vice versa. This scheme of complete or perfect complementarity may, in certain embodiments of the invention, be modified by the possibility of the presence of single or multiple non-complementary bases or stretches of nucleotides within the sense and/or antisense strand(s). Thus, to fall within the notion of a pair of sense and antisense strands, both strands may be completely complementary or may be only partially complementary, e.g., show a complementarity of about 80%, 85%, 90%, 91%, 92%, 93%, 94%, 95%, 96%, 97%, 98%, 99%, 99.5%, or 100% between all nucleotides of both strands or, preferably, between all nucleotides in specific segments as defined herein. Non-complementary bases may comprise one of the nucleotides A, T, G, C, i.e., show a mismatch e.g., between A and G, or T and C, or may comprise any modified nucleoside bases including, for example, modified bases as described in WIPO Standard ST.26. Furthermore, the present invention also envisages complementarity between non-identical nucleic acid molecules, e.g., between a DNA strand and a RNA strand, a DNA strand and a PNA strand, a DNA strand and a CNA strand, etc. It is preferred that the complementarity between strands and, in particular, between segments as defined herein is a complete or 100% complementarity.

The wording “composed of capture probe and a complementary binding probe segment linked to a magnetic bead” as used herein means the binding has that probe segment a complementary overlap with said capture probe. The overlap may, for example, be an overlap of 5, 7, 10, 12, 15, 18, 20, 22, 25, 28 or 30 nucleotides, or any value in between the mentioned values. Also envisaged are longer overlaps. Preferred are short overlaps in the range of 5 to 20 nucleotides. The length of the overlap may further be adjusted in view of hybridization efficiency. The overlap typically is at the 3′ end of the capture probe and at the 5′ end of the binding probe. Within said overlap the matching or complementarity between the complementary bases is preferably 100%. In alternative embodiments, the matching is less than 100%, e.g., 99%, 95%, 90%, 85% or less than 85%.

The “binding probe” as used herein thus comprises a nucleic acid segment which is complementary to the capture probe as defined above. It is further linked to a magnetic bead according to the present invention, as described herein. The linker may be a nucleic acid molecule, which is of a variable length, e.g., 5, 10, 15, 20, 25, 30, 40, 50, 60 or more nucleotides or any value in between these values. The linker may, in certain embodiments, not be of the same type of nucleic acid as the complementary segment and/or the capture probe. It may, for example, be a RNA, DNA, PNA, CNA, HNA, LNA or ANA molecule or a mixture thereof. The molecule in its composite form is understood as “binding molecule” within the context of the present invention.

In specific embodiments, in particular in the context of the usage of Cas9 nucleases, the binding probe may preferably comprise a PAM sequence. The term “PAM sequence” or “protospacer adjacent motif sequence” as used herein refers to a short DNA sequence, e.g., 2-6 nucleotides in length, that follows the DNA region targeted for cleavage by the Cas9 CRISPR system. The PAM sequence is accordingly required for a Cas nuclease to cleave and is generally found 3-4 nucleotides downstream from the cleavage site.

It is particularly preferred that the capture probe and/or the binding molecule comprise a segment of LNA. For example, only the capture probe may comprise in the overlapping segment LNAs, or only the binding molecule may comprise in the overlapping segment LNAs, or, preferably, both molecules may comprise in the overlapping segment LNAs. Further envisaged is the presence of a mixture of LNAs and other nucleotides in the overlapping segment. Also envisaged is, in further embodiments, the capture probe and/or the binding molecule comprise, essentially consist of or consist of LNAs or a percentage of LNAs, e.g., 99, 95, 90, 80, 70, 60, 50, 30, 20, 10% or the like. LNAs are typically considered to stabilize the structure of a nucleic acid. Their presence in the complementary segment thus improves the binding between the capture probe and binding molecule and allows for a broader range of hybridization conditions.

In order to facilitate the formation of this composite form firstly a multitude of capture probes tethered to the substrate is provided. Subsequently, a suitable number of binding molecules, e.g., a number which equals the number of capture probes, is added to the reaction mixture, for example in the reaction zone, chamber or vessel surrounding the substrate, e.g., the cartridge of the f-TIR device. This addition must occur under conditions allowing for the hybridization of the binding molecules to the capture probes

A hybridization and thus complementary binding between the capture probe and the binding probe or binding molecule is facilitated in said reaction zone, chamber or vessel surrounding the substrate, e.g., the cartridge of the f-TIR device, by the presence of a suitable buffer and suitable conditions such as salt concentration, pH and temperature. These conditions may preferably be adjusted to the length of the overlap, the sequence of the molecules and/or the nucleic acid types.

The linker segment of the binding molecule as defined above may hence be provided as single stranded entity or segment outside of the complementary segment. This single stranded segment may accordingly be cleaved by a nuclease, i.e., Cas nuclease, within the context of the present invention, which is capable of cleaving singe stranded nucleic acid molecules. Preferably, Cas12, e.g., Cas12a or Cas12b, which have a trans or collateral strand cleavage activity for ssDNA may be used in case the linker segment is a DNA segment. Similarly, Cas13 which has a has a trans or collateral strand cleavage activity single-stranded for ssRNA may be used in case the linker segment is an RNA segment.

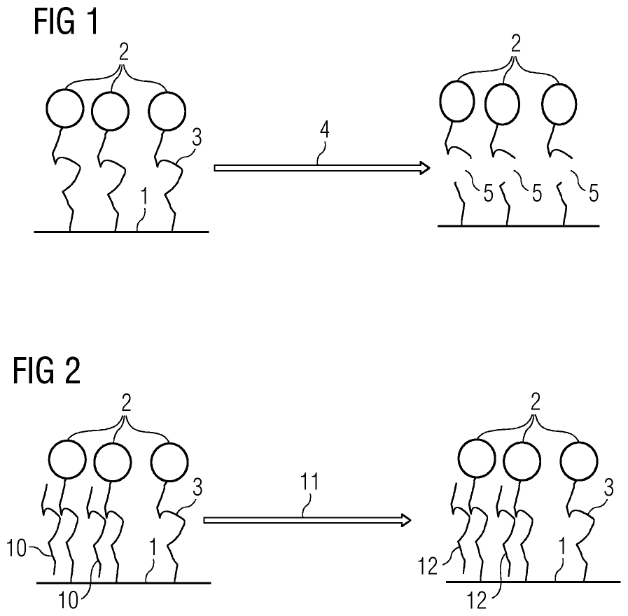

The present invention accordingly envisages, in preferred embodiments, a specific step of the method wherein the nucleic acid detector probe is a single stranded DNA or RNA molecule or comprises a single stranded portion. A complex of a guidance nucleic acid and a not activated nuclease (nuclease complex) as well as a target nucleic acid are added, e.g. flown into a reaction zone, reaction chamber, vessel or any other suitable room which surrounds the substrate, i.e. the sensor surface, wherein said addition results in the provision of a nuclease activity, e.g. of Cas12 or 13, to induce degradation of the single stranded nucleic acid detector probe by an unspecific collateral cut of the nucleic acid detector probe (see also FIG. 1, which depicts this variant of the method for a single stranded nucleic acid detector probe), or degradation of single stranded portion, e.g. of a capture probe hybridized to a binding molecule comprising a single stranded linker by an unspecific collateral cut of the nucleic acid detector probe (see also FIG. 3 which depicts this variant of the method). By adding at the same time, the nuclease complex and the sample which potentially comprises the target nucleic acid an unspecific cleavage of the single stranded binding molecule or single stranded portion occurs which releases the magnetic bead that is thus removed from the substrate or f-TIR surface. The amount of released magnetic beads is directly proportional to the amount of target nucleic acid in the reaction. In the absence of a target nucleic acid no cleavage occurs and all magnetic beads can thus be detected during f-TIR measurements.

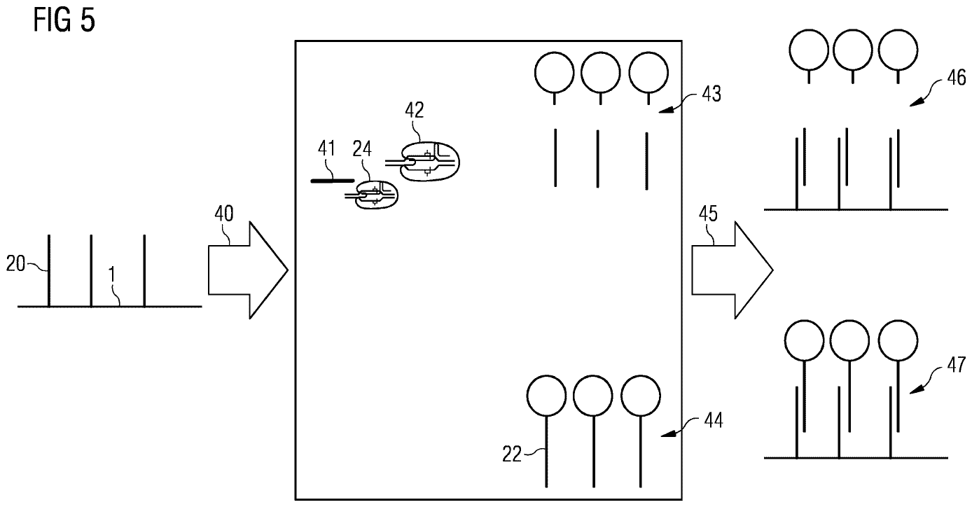

The present invention further envisages, in another preferred embodiment, a specific step (a2) of the method wherein the binding molecule is added, e.g., flown into a reaction zone, reaction chamber, vessel or any other suitable room which surrounds the substrate, i.e., the sensor surface, together with a complex of a guidance nucleic acid and a not activated nuclease (nuclease complex) as well as a target nucleic acid, wherein said addition results in the provision of a nuclease activity, e.g., of Cas12 or 13, to induce degradation of the binding probe (see also FIG. 5, which depicts this variant of the method). By adding at the same time, the nuclease complex and the binding molecule and the target nucleic acid an unspecific cleavage of the then single stranded binding molecule occurs which—due to the absence of the complementary segment after the cleavage—is no longer capable of hybridizing with the capture probe and can thus be removed from the substrate or f-TIR surface. In the absence of a target nucleic acid all added binding molecules can hybridize to the capture probes and thus be detected during f-TIR measurements. Further, in the presence of an excess amount of binding probes or binding molecules, the amount of target nucleic acid can be determined by the number of cleaved binding molecules, since a larger number of target nucleic acids will result in a directly proportional number of cleaved binding molecules and thus unbound magnetic beads. The present invention further envisages, in another preferred embodiment, a variant of the method wherein the nucleic acid detector probe (comprising magnetic beads as described above) is a single stranded molecule, e.g., RNA or preferably DNA. Alternatively, a capture probe is hybridized to a binding molecule, wherein said binding molecule comprises a single stranded linker or stranded portion, e. g., DNA or RNA portion. Importantly, the nucleic acid detector probe or binding molecule comprises as single stranded portion or sequence segment which is complementary to a target nucleic acid. Upon adding of a sample comprising said target nucleic acid, e.g., flown into a reaction zone, reaction chamber, vessel or any other suitable room which surrounds the substrate, i.e., the sensor surface, the nucleic acid may hybridize to the nucleic acid detector probe or the single stranded portion of a molecule. Subsequently, a nuclease complex, e.g., a Cas9 nuclease complex, may be added and becomes activated by the double stranded nucleic acid portion comprising the target nucleic acid. Thereby a specific cleavage of the hybridized nucleic acids is induced, thus resulting in the release of the magnetic bead (see also FIG. 1 or FIG. 4, which depict these variant of the method). The amount of released magnetic beads is directly proportional to the amount of target nucleic acid in the reaction. In the absence of a target nucleic acid no cleavage occurs, and all magnetic beads can thus be detected during f-TIR measurements.

The present invention further envisages, in another preferred embodiment, a specific step of the method wherein the binding molecule is added, e.g. flown into a reaction zone, reaction chamber, vessel or any other suitable room which surrounds the substrate, i.e. the sensor surface, together with a complex of a guidance nucleic acid and a not activated nuclease (nuclease complex) as well as a target nucleic acid, wherein said addition results in the provision of a nuclease activity, e.g. of Cas9. The target nucleic acid may hybridize to the binding molecule, wherein the binding molecule comprises a single stranded portion or sequence segment which is complementary to a target nucleic acid. Thereby a double stranded section is generated which can be recognized by the nuclease complex, resulting in a specific cleavage of the hybridized nucleic acids and subsequently in the release of the magnetic bead. By adding at the same time, the nuclease complex and the binding molecule and the target nucleic acid a cleavage of the binding molecule occurs which—due to the absence of the complementary segment after the cleavage—is no longer capable of hybridizing with the capture probe and can thus be removed from the substrate or f-TIR surface. In the absence of a target nucleic acid all added binding molecules can hybridize to the capture probes and thus be detected during f-TIR measurements. Further, in the presence of an excess number of binding molecules, the amount of target nucleic acid can be determined by the number of cleaved binding molecules, since a larger number of target nucleic acids will result in a directly proportional number of cleaved binding molecules and thus unbound magnetic beads.

The present invention further envisages, in certain embodiments, that in steps (c) and/or (d) as mentioned above, i.e., the steps of providing a complex of a guidance nucleic acid and a not activated nuclease (nuclease complex) and of activating the nuclease complex of step (c) by the addition of a target nucleic acid, the CRISPR-associated protein Csm6 or a functional equivalent thereof is further added. Csm6 is HEPN family ribonuclease which acts as nuclease in the context of CRISPR Cas systems. In specific embodiments, Csm6 specific oligonucleotides may additionally be used which link the magnetic beads to the substrate. Csm6 may be protected by protective oligonucleotides which can be cleaved by a Cas nuclease. Thus, once Cas nucleases are activated, the protective oligonucleotides are cleaved and Csm6 in turn is activated. Csm6 then may cleave the oligonucleotide linkers tethering the magnetic beads to the substrate. Thereby, the magnetic bead may be released. This cascading process may be combined with the herein described nuclease cleavage steps of nucleic acids tethering the magnetic beads. The additional use of the Csm6 activity is assumed to further increase the sensitivity of the method.

It is further envisaged that the method according to the present invention is performed as multiplex method. Accordingly, the detection of target nucleic acids may be performed as detection of a multitude of different target nucleic acids. This detection may be performed in one reaction zone, reaction chamber, vessel or the cartridge of f-TIR device. Preferably, the multitude of different target nucleic acids may be detected at different positions, e.g., in different reaction zones or sub-sections of the substrate, e.g., of the cartridge of the f-TIR device, such as the Atellica VTLi System.

In a particularly preferred embodiment, the multiplexing comprises the use of nucleic acid detector probes directed to 2 to 30 different targets, e.g., 2, 3, 4, 5, 6, 7, 8, 9, 10, 11, 12, 13, 14, 15, 16, 17, 18, 19, 20, 21, 22, 23, 24, 25, 26, 27, 28, 29 or 30 different targets. Particularly preferred are detector probes directed to 2 to 18 different targets. It is further preferred that those nucleic acid detector probes, which are directed to the same target nucleic acid, are provided as spots on the substrate. These spots may, in certain specific embodiments, be analyzed separately or treated in a differential manner depending on the intended research or diagnostic task. Preferably, the spots are analyzed at the same time und undergo the same treatment.

In a further aspect the present invention relates to the use of nucleic acid detector probes and a nuclease complex as defined herein for the detection of a target nucleic by frustrated total internal reflection (f-TIR) measurement. The target nucleic acid may be one as described herein above. It is preferred that the performance of the use of nucleic acid detector probes and a nuclease complex follows the method steps as outlined herein.

Also envisaged is an apparatus performing the above-mentioned method steps. The apparatus may, for example, be composed of different modules which can perform one or more steps of the method of the present invention. These modules may be combined in any suitable fashion, e.g., they may be present in a single place or be separated. Also envisaged is the performance of the method at different points in time and/or in different location. Some steps of the method as define herein may be followed by breaks or pauses, wherein the reagents or products etc. are suitably stored, e.g., in a freezer or a cooling device. In case these steps are performed in specific modules of an apparatus as defined herein, said modules may be used as storage vehicle. The modules may further be used to transport reaction products or reagents to a different location, e. g., different a laboratory etc.

### EXAMPLES

### Example 1

An oropharyngeal throat specimen collection swab is used as a starting sample. Nucleic acid extraction reagents are added to the sample. The extracted nucleic acid is then subjected to amplification method, which can be PCR or any other isothermal amplification method like LAMP or RPA using primers specific to Strep A specific gene. The amplified product is then target nucleic acid of interest.

In one variant, the f-TIR cartridge has a capture probe for Strep A. As a first step, a Strep A binding probe (magnetic bead bound), is flown and bound to the capture probe. Magnetic washes are performed to remove unbound binding probe. A pre-reaction reads out is made (read-out 1). The amplified target nucleic acid is then mixed with the Cas12 enzyme and the complementary sgRNA (CRISPR-Cas12 complex) and applied on the cartridges bound with capture probe. When the target nucleic acid mixed with CRISPR-Cas 12 complex, Cas12 is activated. The activated Cas12 then cleaves the binding probe which is a ssDNA, and the magnetic bead is released, which is washed out during magnetic wash. A post Cas readout is made (read-out 2). The difference of the two read-outs is proportional to the bound probe oligos which is further proportional to the concentration of target analyte nucleic acid present

### Example 2

An oropharyngeal throat specimen collection swab is used as a starting sample. Nucleic acid extraction reagents are added to the sample. The extracted nucleic acid is then subjected to amplification method, which can be PCR or any other isothermal amplification method like LAMP or RPA using primers specific to Strep A specific gene. The amplified product is then the target nucleic acid.

In this variant of the method of the invention, the target nucleic acid is premixed with the binding probe (magnetic bead bound) tethered to a magnetic bead outside the cartridge in a tube (in bulk). The CRISPR-Cas12 complex is then added to this binding probe-target nucleic acid mix. Cas12 is activated, when target nucleic acid is present. The activated Cas 12 then cleaves the binding probe, and the magnetic bead is released. This entire mix is then added to the cartridge surface which has the DNA capture probe. A first read-out (read-out 1) is taken, and the binding step begins. The uncleaved binding probes are still attached to magnetic beads, bind to the capture probe and contribute to signal. A magnetic wash is done to remove the unbound magnetic beads tethered probes. Subsequently, a second read-out (read-out 2) is made. A change in the signal from the baseline (readout 1-readout 2) is proportional to the bound probe oligos which is further proportional to the concentration of target analyte NA present.

The following figures are provided for illustrative purposes. It is thus understood that the figures are not to be construed as limiting. The skilled person in the art will clearly be able to envisage further modifications of the principles laid out herein.

1. A method for the detection of a target nucleic acid, comprising the steps:
(a) providing a multitude of nucleic acid detector probes, each being linked to a magnetic bead and tethered to a substrate;
(b) detecting the number of magnetic beads linked to the substrate by frustrated total internal reflection (f-TIR);
(c) providing a complex of a guidance nucleic acid and a not activated nuclease (nuclease complex);
(d) activating the nuclease complex of step (c) by the addition of a target nucleic acid, wherein said activation results in the provision of a nuclease activity, to induce degradation of the nucleic acid detector probe;
(e) removing unbound magnetic beads;
(f) detecting the number of magnetic beads linked to the substrate by f-TIR;
(g) determining the amount of target nucleic acid in accordance with the ratio of magnetic beads detected in step (b) and step (f).

2. The method of claim 1, wherein the nucleic acid detector probe is composed of a capture probe being tethered to a substrate and a complementary binding probe segment, which is linked to a magnetic bead (binding molecule), wherein said step (a) comprises the sub-steps (a1) of providing a multitude of capture probes tethered to the substrate, and (a2) adding the binding molecule under conditions which allow for hybridization of the binding molecules to the capture probes.

3. The method of claim 2, wherein the capture probe and/or the binding molecule comprises a segment of locked nucleic acids (LNAs).

4. The method of claim 3, wherein said capture probe and said binding molecule comprise LNAs in a complementary segment.

5. The method of claim 1, wherein the nucleic acid detector probe is a single stranded DNA molecule or comprises a single stranded DNA portion, wherein said nucleic acid detector probe comprises a sequence segment which is complementary to the target nucleic acid and wherein the presence of the target nucleic acid being hybridized to the single stranded portion of the nucleic acid detector probe activates the nuclease complex and induces a specific cut of the hybridized nucleic acids.

6. The method of claim 5, wherein the binding probe comprises a PAM sequence.

7. The method of claim 2, wherein in step (a2) the binding molecule is added together with a complex of a guidance nucleic acid and a not activated nuclease (nuclease complex) as well as a target nucleic acid, wherein said addition results in the provision of a nuclease activity, to induce degradation of the binding probe.

8. The method of claim 1, wherein the nucleic acid detector probe is a single stranded DNA or RNA molecule or comprises a single stranded portion, and wherein the presence of the target nucleic acid bound to the nuclease complex activates the nuclease complex and induces an unspecific collateral cut of the nucleic acid detector probe.

9. The method of claim 1, wherein, previous to step (d), the target nucleic acid is amplified.

10. The method of claim 9, wherein said amplification is an isothermal amplification.

11. The method of claim 1, wherein in steps (c) and/or (d) CRISPR-associated protein Csm6 or a functional equivalent thereof is added.

12. The method of claim 1, wherein the nucleic acid detector probes on the substrate are directed to a multitude of different target nucleic acids.

13. The method of claim 12, wherein said nucleic acid detector probes are directed to 2 to 30 different target nucleic acids, and wherein the nucleic acid detector probes directed to the same target nucleic acid are provided as spots on the substrate.

14. The method of claim 5, wherein the nuclease is a Cas9 nuclease or a functional equivalent thereof.

15. The method of claim 7, wherein the nuclease is a Cas12 nuclease or a Cas13 nuclease or a functional equivalent thereof.

16. The method of claim 9, wherein the nuclease is a Cas9 nuclease or a functional equivalent thereof.

17. The method of claim 11, wherein the nuclease is a Cas9 nuclease or a functional equivalent thereof.

18. The method of claim 12, wherein the nuclease is a Cas9 nuclease or a functional equivalent thereof.

19. The method of claim 8, wherein the nuclease is a Cas12 nuclease or a Cas13 nuclease or a functional equivalent thereof.

20. The method of claim 9, wherein the nuclease is a Cas12 nuclease or a Cas13 nuclease or a functional equivalent thereof.
